(19) United States
(12) Patent Application Publication
(10) Pub. No.: US 2023/0281391 A1
(43) Pub. Date: Sep. 7, 2023

(54) SYSTEMS AND METHODS FOR BIOMEDICAL INFORMATION EXTRACTION, ANALYTIC GENERATION AND VISUAL REPRESENTATION THEREOF

(71) Applicant: Sumitomo Pharma Co., Ltd., Chuo-ku (JP)

(72) Inventors: Yoann Mamy Randriamihaja, Brooklyn, NY (US); Mingzhe Tao, Fairfax, VA (US); Mourad Sarrouti, Astoria, NY (US)

(73) Assignee: Sumitomo Pharma Co., Ltd., Chuo-ku (JP)

(21) Appl. No.: 18/118,075

(22) Filed: Mar. 6, 2023

Related U.S. Application Data
(60) Provisional application No. 63/448,648, filed on Feb. 27, 2023.
(60) Provisional application No. 63/345,738, filed on May 25, 2022.
(60) Provisional application No. 63/317,318, filed on Mar. 7, 2022.

(51) Int. Cl. G06F 40/295; G06N 3/0455; G06F 40/166; G06T 11/20; G06F 3/04842

(52) U.S. Cl. CPC G06F 40/295; G06F 3/04842; G06F 40/166; G06N 3/0455; G06T 11/206; G16H 50/70

(57) ABSTRACT

A computer-implemented method, comprising the steps of receiving a plurality of texts, each of the plurality of texts comprising a plurality of syntactic units; extracting, for each of the plurality of syntactic units, at least one subject and/or at least one object; classifying, for each of the plurality of syntactic units, whether each of the plurality of syntactic units comprises a relation; extracting, for each of the plurality of syntactic units, based on the at least one subject, the at least one object, and the syntactic unit, a predicate; normalizing, for each of the plurality of syntactic units, the at least one subject and the at least one object, and appending identifiers to the at least one subject and the at least one object, respectively; and outputting, for each of the syntactic units, a triplet comprising the at least one subject, the at least one object, and the predicate.

### CROSS-REFERENCE TO RELATED APPLICATIONS

The present application claims the benefit of U.S. Pat. Application No. 63/317,318 for TRANSFORMERS FOR RELATION EXTRACTION, filed Mar. 7, 2022, U.S. Pat. Application No. 63/345,738 for INFORMATION EXTRACTION AND KNOWLEDGE GRAPH GENERATION, filed May 25, 2022, and U.S. Pat. Application No. 63/448,648 for SYSTEMS AND METHODS FOR BIOMEDICAL INFORMATION EXTRACTION, ANALYTIC GENERATION AND VISUAL REPRESENTATION THEREOF, filed Feb. 27, 2023, the entire contents of which are incorporated herein by reference in their entirety.

### FIELD OF INVENTION

The present invention is in the field of biomedical information analysis, specifically end-to-end automatic biomedical information extraction and relation extraction thereof.

### INTRODUCTION

Scientific literature provides a rich source of biomedical knowledge (e.g., drug-drug interactions), and due to its rapid growth, it has become increasingly difficult for scientists to keep up to date with the most recent discoveries hidden in literature (Zhang and Lu, 2019; Yadav et al., 2020). Moreover, manual curation of information from biomedical literature is time-consuming, costly, and insufficient to keep up with the rapid growth of such a body of literature (Herrero-Zazo et al., 2013). Hence, there has been growing interest in using natural language processing (NLP) techniques for relation extraction (RE) between biomedical entities from texts.

Traditionally, a variety of approaches based on pretrained language models such as BERT (Devlin et al., 2019) and other variants may have been utilized for various NLP tasks, such as relation extraction, question answering (Sarrouti et al., 2021b), text summarization (Goodwin et al., 2020; Yadav et al., 2021), and misinformation detection (Sarrouti et al., 2021a). In particular, traditional methods may utilize RE with classification-based encoder-only pretrained transformers (i.e., BERT and variants) (Lee et al., 2019; Peng et al., 2019a; Gu et al., 2022).

Accordingly, in an attempt to harness information within biomedical literature, in the past there was a surge in interest from the NLP community to automatically extract relations between biomedical entities (i.e., proteins, gene, diseases, etc.) from said biomedical literature (Krallinger et al., 2008; Segura-Bedmar et al., 2013; Krallinger et al., 2017; Miranda et al., 2021). Consequently, with the increased use of pretrained language models, there is a need to explore techniques based on transformers for extracting the relationships between entities from biomedical literature (Thillaisundaram and Togia, 2019; Wei et al., 2019; Hebbar and Xie, 2021; Hiai et al., 2021; Liu et al., 2021; Zhou et al., 2021; Su et al., 2021; Chang et al., 2021; Weber et al., 2021). Such conventional systems have primarily been configured with encoder-only transformers, such as BERT (Devlin et al., 2019) and its variants like SciBERT (Beltagy et al., 2019), BioBERT (Lee et al., 2019), and PubMedBERT (Gu et al., 2022). Accordingly, it would be desirable to provide improved RE methods. Unlike RE with classification-based encoder-only transformers of conventional methods, RE with encoder-decoder transformers may improve biomedical text analysis. Thus, it would be desirable to provide systems and methods utilizing encoder-decoder-based transformers, such as T5, (Raffel et al., 2020) which has shown strong performance in various NLP tasks such as question answering and text summarization.

Accordingly, it would be desirable to provide systems and methods configured for improved relation extraction from biomedical texts, specifically to fuel an improved Named-entity recognition (NER) Pipeline workflow. It would be further desirable to provide systems and methods configured to generate and display visual representations of extracted biomedical information.

### SUMMARY

In accordance with the present disclosure, the following items are provided.

(Item 1). A computer-implemented method, comprising the steps of:
receiving a plurality of texts, each of the plurality of texts comprising a plurality of syntactic units;
extracting, via a NER model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate;
normalizing, for each of the plurality of syntactic units, the at least one subject and the at least one object, and appending a subject identifier and an object identifier to the at least one subject and the at least one object, respectively; and
outputting, for each of the plurality of syntactic units, a triplet comprising the at least one subject, the at least one object, and the predicate.

(Item 2). The computer-implemented method of Item 1, further comprising the step of: generating, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

(Item 3). The computer-implemented method of Item 2, wherein each of the plurality of nodes are configurable in a plurality of nodal indications, wherein each of the plurality of nodal indications correspond to one of a plurality of entity categories.

(Item 4). The computer-implemented method of Item 2 or Item 3, wherein each of the plurality of edges are configurable in a plurality of edge indications, wherein each of the edge indications correspond to one of a plurality of relationship categories.

(Item 5). The computer-implemented method of any one of Items 2 to 4, further comprising the steps of:
receiving, via a client device, a selection input corresponding to one of the plurality of nodes or the plurality of edges;
generating a summary representation comprising at least one of the subject, the object, the predicate, the syntactic unit, or the text, based on the selection input; and
displaying, via the client device, the summary representation.

(Item 6). The computer-implemented method of any one of Items 1 to 5, wherein the predicate extraction model is an encoder-decoder model.

(Item 7). The computer-implemented method of Item 6, wherein the encoder-decoder model is tuned via a multi-task fine-tuning.

(Item 8). The computer-implemented method of Item 6 or Item 7, wherein the encoder-decoder model is trained on a plurality of biomedical texts.

(Item 9). The computer-implemented method of any one of Items 1 to 8, wherein the classification model is an encoder-only model.

(Item 10). The computer-implemented method of any one of Items 1 to 8, wherein the classification model is an encoder-decoder model.

(Item 11). A system, comprising:
a server comprising at least one server processor, at least one server database, at least one server memory comprising computer-executable server instructions which, when executed by the at least one server processor, cause the server to:
   receive a plurality of texts, each of the plurality of texts comprising a plurality of syntactic units;
   extract, via a NER model, for each of the plurality of syntactic units, at least one subject and/or at least one object;
   classify, via a classification model, for each of the plurality of syntactic units, whether each of the plurality of syntactic units comprises a relation;
   extract, via a predicate extraction model, for each of the plurality of syntactic units, based on the at least one subject, the at least one object, and the corresponding syntactic unit, a predicate;
   normalize, for each of the plurality of syntactic units, the at least one subject and the at least one object, and appending a subject identifier and an object identifier to the at least one subject and the at least one object, respectively;
   output, for each of the plurality of syntactic units, a triplet comprising the at least one subject, the at least one object, and the predicate.

(Item 12). The system of Item 11, the computer-executable server instructions which, when executed by the at least one server processor, further cause the server to:
generate, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

(Item 13). The system of Item 12, wherein each of the plurality of nodes are configurable in a plurality of nodal indications, wherein each of the plurality of nodal indications correspond to one of a plurality of entity categories.

(Item 14). The system of Item 12 or Item 13, wherein each of the plurality of edges are configurable in a plurality of edge indications, wherein each of the edge indications correspond to one of a plurality of relationship categories.

(Item 15). The system of any one of Items 12 to 14, further comprising:
a client device in bidirectional communication with the server, the client device comprising at least one device processor, at least one display, at least one device memory comprising computer-executable device instructions which, when executed by the at least one device processor, cause the client device to:
   receive, via the client device, a selection input corresponding to one of the plurality of nodes or the plurality of edges; and
   display, via the client device, a summary representation, and
the computer-executable server instructions which, when executed by the at least one server processor, further cause the server to:
   generate the summary representation comprising at least one of the subject, the object, the predicate, the syntactic unit, or the text, based on the selection input.

(Item 16). The system of any one of Items 11 to 15, wherein the predicate extraction model is an encoder-decoder model.

(Item 17). The system of Item 16, wherein the encoder-decoder model is tuned via multi-task fine-tuning.

(Item 18). The system of Item 16 or Item 17, wherein the encoder-decoder model is trained on a plurality of biomedical texts.

(Item 19). The system of any one of Items 11 to 18, wherein the classification model is an encoder-only model.

(Item 20). The system of any one of Items 11 to 18, wherein the classification model is an encoder-decoder model.

(Item 21). A non-transitory computer readable medium having instructions stored thereon that, when executed by a processing device, cause the processing device to carry out an operation of triplet extraction and visualization generation between a server and a client device, the operation comprising:
receiving a plurality of texts, each of the plurality of texts comprising a plurality of syntactic units;
extracting, via a NER model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate;
normalizing, for each of the plurality of syntactic units, the at least one subject and the at least one object, and appending a subject identifier and an object identifier to the at least one subject and the at least one object, respectively; and
outputting, for each of the plurality of syntactic units, a triplet comprising the at least one subject, the at least one object, and the predicate.

(Item 22). The non-transitory computer readable medium of Item 21, the operation further comprising:
generating, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

(Item 23). The non-transitory computer readable medium of Item 22, wherein each of the plurality of nodes are configurable in a plurality of nodal indications, wherein each of the plurality of nodal indications correspond to one of a plurality of entity categories.

(Item 24). The non-transitory computer readable medium of Item 22 or Item 23, wherein each of the plurality of edges are configurable in a plurality of edge indications, wherein each of the edge indications correspond to one of a plurality of relationship categories.

(Item 25). The non-transitory computer readable medium of any one of Items 22 to 24, the operation further comprising:
receiving, via the client device, a selection input corresponding to one of the plurality of nodes or the plurality of edges;
generating a summary representation comprising at least one of the subject, the object, the predicate, the syntactic unit, or the text, based on the selection input; and
displaying, via the client device, the summary representation.

(Item 26). The non-transitory computer readable medium of any one of Items 21 to 25, wherein the predicate extraction model is an encoder-decoder model.

(Item 27). The non-transitory computer readable medium of Item 26, wherein the encoder-decoder model is tuned via a multi-task fine-tuning.

(Item 28). The non-transitory computer readable medium of Item 26 or Item 27, wherein the encoder-decoder model is trained on a plurality of biomedical texts.

(Item 29). The non-transitory computer readable medium of any one of Items 21 to 28, wherein the classification model is an encoder-only model.

(Item 30). The non-transitory computer readable medium of any one of Items 21 to 28, wherein the classification model is an encoder-decoder model.

(Item 31). A non-transitory computer readable medium having instructions stored thereon that, when executed by a processing device, cause the processing device to carry out an operation of triplet extraction and visualization generation between a server and a client device, the operation comprising:
receiving a plurality of texts, each of the plurality of texts comprising a plurality of syntactic units;
extracting, via a NER model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate, wherein the predicate extraction model is an encoder-decoder model;
normalizing, for each of the plurality of syntactic units, the at least one subject and the at least one object, and appending a subject identifier and an object identifier to the at least one subject and the at least one object, respectively;
outputting, for each of the plurality of syntactic units, a triplet comprising the at least one subject, the at least one object, and the predicate; and
generating, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type,
wherein the relationship type is based on at least the predicate of each of the triplets derived from the plurality of texts,
wherein each of the plurality of nodes are configurable in a plurality of nodal indications, each of the plurality of nodal indications corresponding to one of a plurality of entity categories, and wherein each of the plurality of edges are configurable in a plurality of edge indications, each of the edge indications corresponding to one of a plurality of relationship categories.

(Item 32). A system, comprising:
a server comprising at least one server processor, at least one server database, at least one server memory comprising computer-executable server instructions which, when executed by the at least one server processor, cause the server to:
   receive a plurality of texts, each of the plurality of texts comprising a plurality of syntactic units;
   extract, via a NER model, for each of the plurality of syntactic units, at least one subject and/or at least one object;
   classify, via a classification model, for each of the plurality of syntactic units, whether each of the plurality of syntactic units comprises a relation,
   wherein the classification model is an encoder-decoder model;
   extract, via a predicate extraction model, for each of the plurality of syntactic units, based on the at least one subject, the at least one object, and the corresponding syntactic unit, a predicate;
   normalize, for each of the plurality of syntactic units, the at least one subject and the at least one object, and appending a subject identifier and an object identifier to the at least one subject and the at least one object, respectively;
   output, for each of the plurality of syntactic units, a triplet comprising the at least one subject, the at least one object, and the predicate; and
   generate, based on the triplets derived from the plurality of texts, a knowledge graph comprising a plurality of nodes and a plurality of edges,
   wherein each of the plurality of nodes corresponds to at least one subject or at least one object of each of the triplets derived from the plurality of texts,
   wherein each of the plurality of edges corresponds to a relationship type, and
   wherein the relationship type is based on at least the predicate of each of the triplets derived from the plurality of texts;
   generate a summary representation comprising at least one of the subject, the object, the predicate, the syntactic unit, or the text, based on a selection input; and
   a client device in bidirectional communication with the server, the client device comprising at least one device processor, at least one display, at least one device memory comprising computer-executable device instructions which, when executed by the at least one device processor, cause the client device to:
      receive, via the client device, the selection input corresponding to one of the plurality of nodes or the plurality of edges; and
      display, via the client device, the summary representation.

(Item 33). A computer-implemented method, comprising the steps of:
receiving a plurality of texts, each of the plurality of texts comprising a plurality of syntactic units;
extracting, via a NER model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate, wherein the predicate extraction model is an encoder-decoder model,
wherein the encoder-decoder model is tuned via a multi-task fine-tuning, and
wherein the encoder-decoder model is trained on a plurality of biomedical texts;
normalizing, for each of the plurality of syntactic units, the at least one subject and the at least one object, and appending a subject identifier and an object identifier to the at least one subject and the at least one object, respectively;
outputting, for each of the plurality of syntactic units, a triplet comprising the at least one subject, the at least one object, and the predicate;
generating, based on the triplets derived from the plurality of texts, a knowledge graph;
receiving, via a client device, a selection input corresponding to one of the plurality of nodes or the plurality of edges;
generating a summary representation comprising at least one of the subject, the object, the predicate, the syntactic unit, or the text, based on the selection input; and
displaying, via the client device, the summary representation.

(Item 34). A non-transitory computer readable medium having instructions stored thereon that, when executed by a processing device, cause the processing device to carry out an operation of triplet extraction and visualization generation between a server and a client device, the operation comprising:
receiving a plurality of texts, each of the plurality of texts comprising a plurality of syntactic units;
extracting, for each of the plurality of syntactic units, at least one subject and/or at least one object;
classifying, for each of the plurality of syntactic units, whether each of the plurality of syntactic units comprises a relation;
extracting, for each of the plurality of syntactic units, a predicate; and
outputting, for each of the plurality of syntactic units, a triplet comprising the at least one subject, the at least one object, and the predicate.

Additional aspects related to this disclosure are set forth, in part, in the description which follows, and, in part, will be obvious from the description, or may be learned by practice of this disclosure.

It is to be understood that both the forgoing and the following descriptions are exemplary and explanatory only and are not intended to limit the claimed disclosure or application thereof in any manner whatsoever.

### BRIEF DESCRIPTION OF THE DRAWINGS

The incorporated drawings, which are incorporated in and constitute a part of this specification exemplify the aspects of the present disclosure and, together with the description, explain and illustrate principles of this disclosure.

FIG. 1 illustrates a block diagram of a distributed computer system that can implement one or more aspects of an embodiment of the present invention.

FIG. 2 illustrates a block diagram of an electronic device that can implement one or more aspects of an embodiment of the invention.

FIG. 3 is a diagram illustrating an example of a NER pipeline workflow.

FIG. 4 is a diagram illustrating an example of an end-to-end triplet extraction from biomedical texts.

FIG. 5 is a diagram illustrating a simplified example of a biomedical knowledge graph.

FIG. 6 is a diagram illustrating multi-task learning for biomedical relation extraction.

FIG. 7 is a flowchart illustrating an embodiment of triplet extraction and knowledge graph generation.

### DETAILED DESCRIPTION

In the following detailed description, reference will be made to the accompanying drawing(s), in which identical functional elements are designated with like numerals. The aforementioned accompanying drawings show by way of illustration, and not by way of limitation, specific aspects, and implementations consistent with principles of this disclosure. These implementations are described in sufficient detail to enable those skilled in the art to practice the disclosure and it is to be understood that other implementations may be utilized and that structural changes and/or substitutions of various elements may be made without departing from the scope and spirit of this disclosure. The following detailed description is, therefore, not to be construed in a limited sense.

It is noted that description herein is not intended as an extensive overview, and as such, concepts may be simplified in the interests of clarity and brevity.

All documents mentioned in this application are hereby incorporated by reference in their entirety. Any process described in this application may be performed in any order and may omit any of the steps in the process. Processes may also be combined with other processes or steps of other processes.

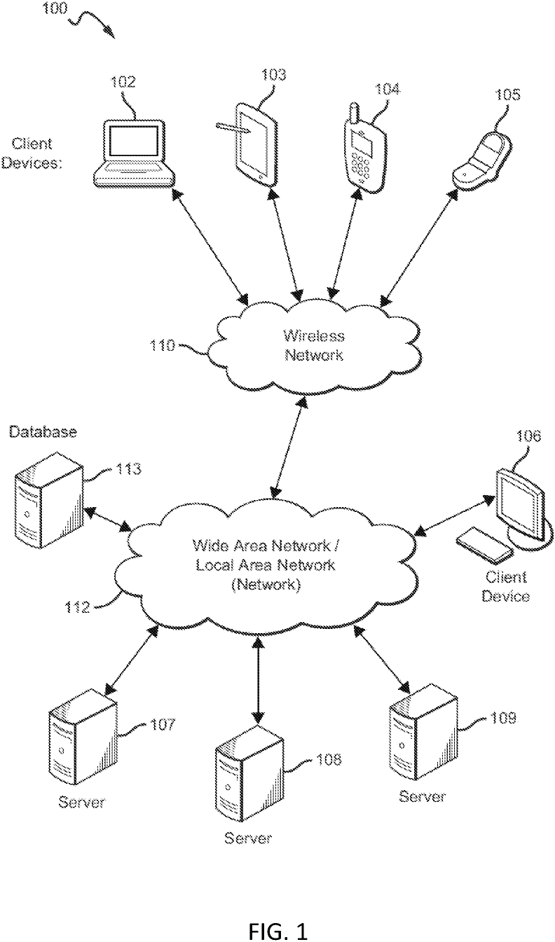

FIG. 1 illustrates components of one embodiment of an environment in which the invention may be practiced. Not all of the components may be required to practice the invention, and variations in the arrangement and type of the components may be made without departing from the spirit or scope of the invention. As shown, the system 100 includes one or more Local Area Networks (“LANs”)/Wide Area Networks (“WANs”) 112, one or more wireless networks 110, one or more wired or wireless client devices 106, mobile or other wireless client devices 102-105, servers 107-109, and may include or communicate with one or more data stores or databases. Various of the client devices 102-106 may include, for example, desktop computers, laptop computers, set top boxes, tablets, cell phones, smart phones, smart speakers, wearable devices (such as the Apple Watch) and the like. Servers 107-109 can include, for example, one or more application servers, content servers, search servers, and the like.

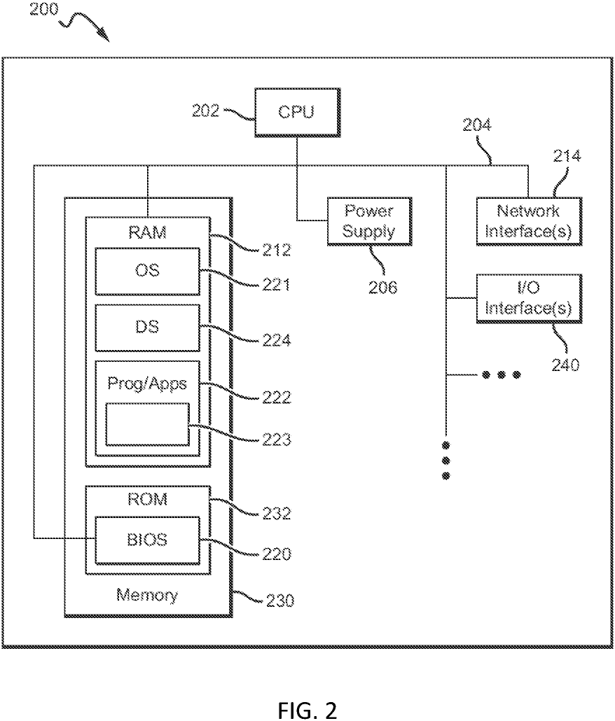

FIG. 2 illustrates a block diagram of an electronic device 200 that can implement one or more aspects of an apparatus, system and method for validating and correcting user information (the “Engine”) according to one embodiment of the invention. Instances of the electronic device 200 may include servers, e.g., servers 107-109, and client devices, e.g., client devices 102-106. In general, the electronic device 200 can include a processor/CPU 202, memory 230, a power supply 206, and input/output (I/O) components/devices 240, e.g., microphones, speakers, displays, touchscreens, keyboards, mice, keypads, microscopes, GPS components, cameras, heart rate sensors, light sensors, accelerometers, targeted biometric sensors, etc., which may be operable, for example, to provide graphical user interfaces or text user interfaces.

A user may provide input via a touchscreen of an electronic device 200. A touchscreen may determine whether a user is providing input by, for example, determining whether the user is touching the touchscreen with a part of the user’s body such as his or her fingers. The electronic device 200 can also include a communications bus 204 that connects the aforementioned elements of the electronic device 200. Network interfaces 214 can include a receiver and a transmitter (or transceiver), and one or more antennas for wireless communications.

The processor 202 can include one or more of any type of processing device, e.g., a Central Processing Unit (CPU), and a Graphics Processing Unit (GPU). Also, for example, the processor can be central processing logic, or other logic, may include hardware, firmware, software, or combinations thereof, to perform one or more functions or actions, or to cause one or more functions or actions from one or more other components. Also, based on a desired application or need, central processing logic, or other logic, may include, for example, a software-controlled microprocessor, discrete logic, e.g., an Application Specific Integrated Circuit (ASIC), a programmable/programmed logic device, memory device containing instructions, etc., or combinatorial logic embodied in hardware. Furthermore, logic may also be fully embodied as software.

The memory 230, which can include Random Access Memory (RAM) 212 and Read Only Memory (ROM) 232, can be enabled by one or more of any type of memory device, e.g., a primary (directly accessible by the CPU) or secondary (indirectly accessible by the CPU) storage device (e.g., flash memory, magnetic disk, optical disk, and the like). The RAM can include an operating system 221, data storage 224, which may include one or more databases, and programs and/or applications 222, which can include, for example, software aspects of the program 223. The ROM 232 can also include Basic Input/Output System (BIOS) 220 of the electronic device.

Software aspects of the program 223 are intended to broadly include or represent all programming, applications, algorithms, models, software and other tools necessary to implement or facilitate methods and systems according to embodiments of the invention. The elements may exist on a single computer or be distributed among multiple computers, servers, devices or entities.

The power supply 206 contains one or more power components, and facilitates supply and management of power to the electronic device 200.

The input/output components, including Input/Output (I/O) interfaces 240, can include, for example, any interfaces for facilitating communication between any components of the electronic device 200, components of external devices (e.g., components of other devices of the network or system 100), and end users. For example, such components can include a network card that may be an integration of a receiver, a transmitter, a transceiver, and one or more input/output interfaces. A network card, for example, can facilitate wired or wireless communication with other devices of a network. In cases of wireless communication, an antenna can facilitate such communication. Also, some of the input/output interfaces 240 and the bus 204 can facilitate communication between components of the electronic device 200, and in an example can ease processing performed by the processor 202.

Where the electronic device 200 is a server, it can include a computing device that can be capable of sending or receiving signals, e.g., via a wired or wireless network, or may be capable of processing or storing signals, e.g., in memory as physical memory states. The server may be an application server that includes a configuration to provide one or more applications, e.g., aspects of the Engine, via a network to another device. Also, an application server may, for example, host a web site that can provide a user interface for administration of example aspects of the Engine.

Any computing device capable of sending, receiving, and processing data over a wired and/or a wireless network may act as a server, such as in facilitating aspects of implementations of the Engine. Thus, devices acting as a server may include devices such as dedicated rack-mounted servers, desktop computers, laptop computers, set top boxes, integrated devices combining one or more of the preceding devices, and the like.

Servers may vary widely in configuration and capabilities, but they generally include one or more central processing units, memory, mass data storage, a power supply, wired or wireless network interfaces, input/output interfaces, and an operating system such as Windows Server, Mac OS X, Unix, Linux, FreeBSD, and the like.

A server may include, for example, a device that is configured, or includes a configuration, to provide data or content via one or more networks to another device, such as in facilitating aspects of an example apparatus, system and method of the Engine. One or more servers may, for example, be used in hosting a Web site, such as the web site www.microsoft.com. One or more servers may host a variety of sites, such as, for example, business sites, informational sites, social networking sites, educational sites, wikis, financial sites, government sites, personal sites, and the like.

Servers may also, for example, provide a variety of services, such as Web services, third-party services, audio services, video services, email services, HTTP or HTTPS services, Instant Messaging (IM) services, Short Message Service (SMS) services, Multimedia Messaging Service (MMS) services, File Transfer Protocol (FTP) services, Voice Over IP (VOIP) services, calendaring services, phone services, and the like, all of which may work in conjunction with example aspects of an example systems and methods for the apparatus, system and method embodying the Engine. Content may include, for example, text, images, audio, video, and the like.

In example aspects of the apparatus, system and method embodying the Engine, client devices may include, for example, any computing device capable of sending and receiving data over a wired and/or a wireless network. Such client devices may include desktop computers as well as portable devices such as cellular telephones, smart phones, display pagers, Radio Frequency (RF) devices, Infrared (IR) devices, Personal Digital Assistants (PDAs), handheld computers, GPS-enabled devices tablet computers, sensor-equipped devices, laptop computers, set top boxes, wearable computers such as the Apple Watch and Fitbit, integrated devices combining one or more of the preceding devices, and the like.

Client devices such as client devices 102-106, as may be used in an example apparatus, system and method embodying the Engine, may range widely in terms of capabilities and features. For example, a cell phone, smart phone or tablet may have a numeric keypad and a few lines of monochrome Liquid-Crystal Display (LCD) display on which only text may be displayed. In another example, a Web-enabled client device may have a physical or virtual keyboard, data storage (such as flash memory or SD cards), accelerometers, gyroscopes, respiration sensors, body movement sensors, proximity sensors, motion sensors, ambient light sensors, moisture sensors, temperature sensors, compass, barometer, fingerprint sensor, face identification sensor using the camera, pulse sensors, heart rate variability (HRV) sensors, beats per minute (BPM) heart rate sensors, microphones (sound sensors), speakers, GPS or other location-aware capability, and a 2D or 3D touch-sensitive color screen on which both text and graphics may be displayed. In some embodiments multiple client devices may be used to collect a combination of data. For example, a smart phone may be used to collect movement data via an accelerometer and/or gyroscope and a smart watch (such as the Apple Watch) may be used to collect heart rate data. The multiple client devices (such as a smart phone and a smart watch) may be communicatively coupled.

Client devices, such as client devices 102-106, for example, as may be used in an example apparatus, system and method implementing the Engine, may run a variety of operating systems, including personal computer operating systems such as Windows, iOS or Linux, and mobile operating systems such as iOS, Android, Windows Mobile, and the like. Client devices may be used to run one or more applications that are configured to send or receive data from another computing device. Client applications may provide and receive textual content, multimedia information, and the like. Client applications may perform actions such as browsing webpages, using a web search engine, interacting with various apps stored on a smart phone, sending and receiving messages via email, SMS, or MMS, playing games (such as fantasy sports leagues), receiving advertising, watching locally stored or streamed video, or participating in social networks.

In example aspects of the apparatus, system and method implementing the Engine, one or more networks, such as networks 110 or 112, for example, may couple servers and client devices with other computing devices, including through wireless network to client devices. A network may be enabled to employ any form of computer readable media for communicating information from one electronic device to another. The computer readable media may be non-transitory. Thus, in various embodiments, a non-transitory computer readable medium may comprise instructions stored thereon that, when executed by a processing device, cause the processing device to carry out an operation (e.g., triplet extraction and visualization generation). In such an embodiment, the operation may be carried out on a singular device or between multiple devices (e.g., a server and a client device). A network may include the Internet in addition to Local Area Networks (LANs), Wide Area Networks (WANs), direct connections, such as through a Universal Serial Bus (USB) port, other forms of computer-readable media (computer-readable memories), or any combination thereof. On an interconnected set of LANs, including those based on differing architectures and protocols, a router acts as a link between LANs, enabling data to be sent from one to another.

Communication links within LANs may include twisted wire pair or coaxial cable, while communication links between networks may utilize analog telephone lines, cable lines, optical lines, full or fractional dedicated digital lines including T1, T2, T3, and T4, Integrated Services Digital Networks (ISDNs), Digital Subscriber Lines (DSLs), wireless links including satellite links, optic fiber links, or other communications links known to those skilled in the art. Furthermore, remote computers and other related electronic devices could be remotely connected to either LANs or WANs via a modem and a telephone link.

A wireless network, such as wireless network 110, as in an example apparatus, system and method implementing the Engine, may couple devices with a network. A wireless network may employ stand-alone ad-hoc networks, mesh networks, Wireless LAN (WLAN) networks, cellular networks, and the like.

A wireless network may further include an autonomous system of terminals, gateways, routers, or the like connected by wireless radio links, or the like. These connectors may be configured to move freely and randomly and organize themselves arbitrarily, such that the topology of wireless network may change rapidly. A wireless network may further employ a plurality of access technologies including 2nd (2G), 3rd (3G), 4th (4G) generation, Long Term Evolution (LTE) radio access for cellular systems, WLAN, Wireless Router (WR) mesh, and the like. Access technologies such as 2G, 2.5G, 3G, 4G, and future access networks may enable wide area coverage for client devices, such as client devices with various degrees of mobility. For example, a wireless network may enable a radio connection through a radio network access technology such as Global System for Mobile communication (GSM), Universal Mobile Telecommunications System (UMTS), General Packet Radio Services (GPRS), Enhanced Data GSM Environment (EDGE), 3GPP Long Term Evolution (LTE), LTE Advanced, Wideband Code Division Multiple Access (WCDMA), Bluetooth, 802.11b/g/n, and the like. A wireless network may include virtually any wireless communication mechanism by which information may travel between client devices and another computing device, network, and the like.

Internet Protocol (IP) may be used for transmitting data communication packets over a network of participating digital communication networks, and may include protocols such as TCP/IP, UDP, DECnet, NetBEUI, IPX, Appletalk, and the like. Versions of the Internet Protocol include IPv4 and IPv6. The Internet includes local area networks (LANs), Wide Area Networks (WANs), wireless networks, and long-haul public networks that may allow packets to be communicated between the local area networks. The packets may be transmitted between nodes in the network to sites each of which has a unique local network address. A data communication packet may be sent through the Internet from a user site via an access node connected to the Internet. The packet may be forwarded through the network nodes to any target site connected to the network provided that the site address of the target site is included in a header of the packet. Each packet communicated over the Internet may be routed via a path determined by gateways and servers that switch the packet according to the target address and the availability of a network path to connect to the target site.

The header of the packet may include, for example, the source port (16 bits), destination port (16 bits), sequence number (32 bits), acknowledgement number (32 bits), data offset (4 bits), reserved (6 bits), checksum (16 bits), urgent pointer (16 bits), options (variable number of bits in multiple of 8 bits in length), padding (may be composed of all zeros and includes a number of bits such that the header ends on a 32 bit boundary). The number of bits for each of the above may also be higher or lower.

A “content delivery network” or “content distribution network” (CDN), as may be used in an example apparatus, system and method implementing the Engine, generally refers to a distributed computer system that comprises a collection of autonomous computers linked by a network or networks, together with the software, systems, protocols and techniques designed to facilitate various services, such as the storage, caching, or transmission of content, streaming media and applications on behalf of content providers. Such services may make use of ancillary technologies including, but not limited to, “cloud computing,” distributed storage, DNS request handling, provisioning, data monitoring and reporting, content targeting, personalization, and business intelligence. A CDN may also enable an entity to operate and/or manage a third party’s web site infrastructure, in whole or in part, on the third party’s behalf.

A Peer-to-Peer (or P2P) computer network relies primarily on the computing power and bandwidth of the participants in the network rather than concentrating it in a given set of dedicated servers. P2P networks are typically used for connecting nodes via largely ad hoc connections. A pure peer-to-peer network does not have a notion of clients or servers, but only equal peer nodes that simultaneously function as both “clients” and “servers” to the other nodes on the network.

Embodiments of the present invention include apparatuses, systems, and methods implementing the Engine. Embodiments of the present invention may be implemented on one or more of client devices 102-106, which are communicatively coupled to servers including servers 107-109. Moreover, client devices 102-106 may be communicatively (wirelessly or wired) coupled to one another. In particular, software aspects of the Engine may be implemented in the program 223. The program 223 may be implemented on one or more client devices 102-106, one or more servers 107-109, and 113, or a combination of one or more client devices 102-106, and one or more servers 107-109 and 113.

In an embodiment, the system may receive, process, generate and/or store time series data. The system may include an application programming interface (API). The API may include an API subsystem. The API subsystem may allow a data source to access data. The API subsystem may allow a third-party data source to send the data. In one example, the third-party data source may send JavaScript Object Notation (“JSON”)-encoded object data. In an embodiment, the object data may be encoded as XML-encoded object data, query parameter encoded object data, or byte-encoded object data.

The present disclosure relates to systems and methods for biomedical information analysis, specifically end-to-end automatic biomedical information extraction and relation extraction thereof. In various embodiments, an end-to-end automatic biomedical information extraction system generates nodes and edges of a knowledge graph from narrative text sources.

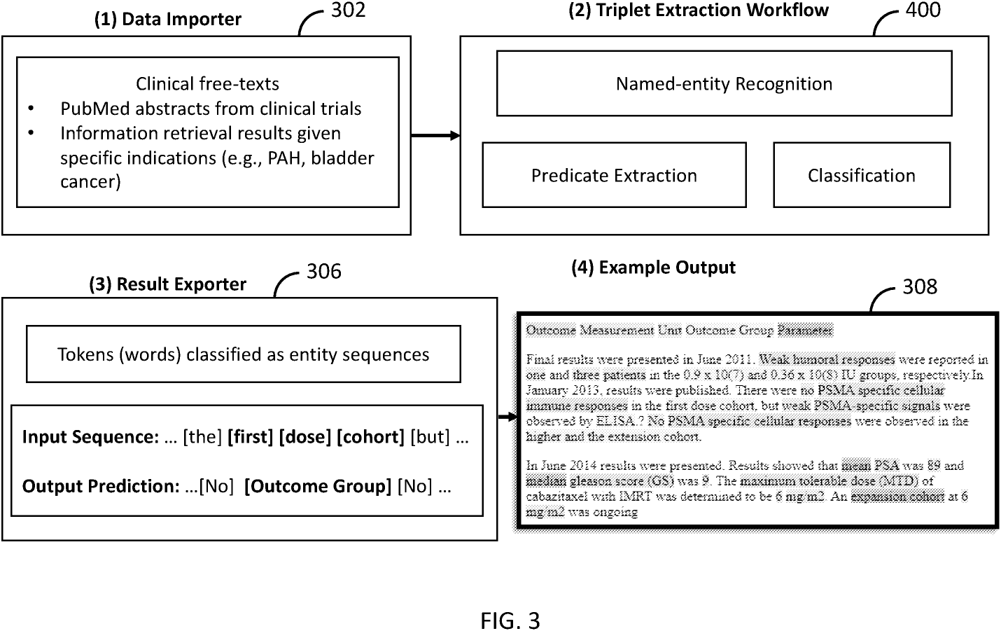

A Named-entity recognition (NER) component may be a component configured to identify biomedical entities such as gene and disease names from narrative free texts. In one embodiment, a pipeline may utilize sequence labeling paired with contextual representation using fine-tuned language models. The output of such a pipeline may contain meaningful entities and their corresponding entity categories. FIG. 3 is a diagram illustrating an example of a NER pipeline workflow. The NER pipeline 300 may include one or more components or modules. For example, as shown in FIG. 3, the NER pipeline 300 may include a data importer module 302, a triplet extraction workflow 400, a Result Exporter 306, and/or an Output module 308. However, in various embodiments, the NER pipeline 300 may include any number and/or combination of components or modules. Further, the recited purposes and/or functionalities of each of the aforementioned components should not be interpreted as limiting, as such purposes and/or functionalities may be practiced by other components described in this disclosure or those contemplated by a person of ordinary skill in the art.

In an embodiment, database 113 is configured to store content items that are retrieved by the data importer module 302. Such content items may include, but are not limited to, text-based documents (e.g., scientific articles or publications, press releases, news articles, books, websites converted into documents, and any other types of documents), images, audio files, video files, tabular files, slide presentation files, medical health records, laboratory tests, genomic data, digital representations of people, and any other types of content items that can be represented digitally. In one embodiment, content items may include clinical free texts, such as, but not limited to, PubMed abstracts or other online literature, from clinical trials and/or information retrieval results given specific indications (e.g., PAH, bladder cancer, etc.). For the purposes of this disclosure, “free text” or “narrative free text” may refer to unstructured texts that contain narrative sentences or other natural language components (i.e., not immediately comprehensible by machines); and “clinical free texts” may refer to a category of free text comprising emphasis on clinically-relevant topics. In some embodiments, database 113 spans multiple data sources (e.g., multiple Internet sources providing documents). In various embodiments, database 113 is a structured set of data held in one or more computers and/or storage devices. In one embodiment, texts may be imported to the importer module 302 and/or the database 113 based on instructions (i.e., search terms or specific indications) indicated by a user on a client device 102-106. Accordingly, database 113 may be a standalone component and/or the contents thereof may exist on any of the devices described herein (e.g., client devices 102-106 or servers 107-109).

In one embodiment, the pipeline 300 may include an NEE API module, comprising a NER pipeline and/or a contextual representation using a fine-tuned language model, wherein said fine-tuned language model may incorporate a recurrent neural network, for example, obtained from the combination of a long short-term memory (LSTM) and a conditional random field (CRF). Such a model may be configured to function as a sequence labeler.

The triplet extraction workflow 400 may be in informatic communication with the importer module 302 and/or the result exporter 306. The triplet extraction workflow 400 is described in further detail below.

The results exporter module 306 may evaluate tokens (i.e., words) classified as entity sequences. Thus, the results exporter module 306 may be adapted to structure, modify, or otherwise process results (i.e., extracted entities), such that the results are presented in a usable format to the example output 308. In an embodiment, the example output 308 may include the original text and/or a summary of the original text, wherein portions of the text identified as entities are indicated (e.g., via highlighting or other visual representation). Accordingly, each entity type may correspond to a particular color or other visual representation, such that entity values and types are distinguishable in the example output 308. In another embodiment, the example output 308 may include and/or may communicate with the knowledge graph 500.

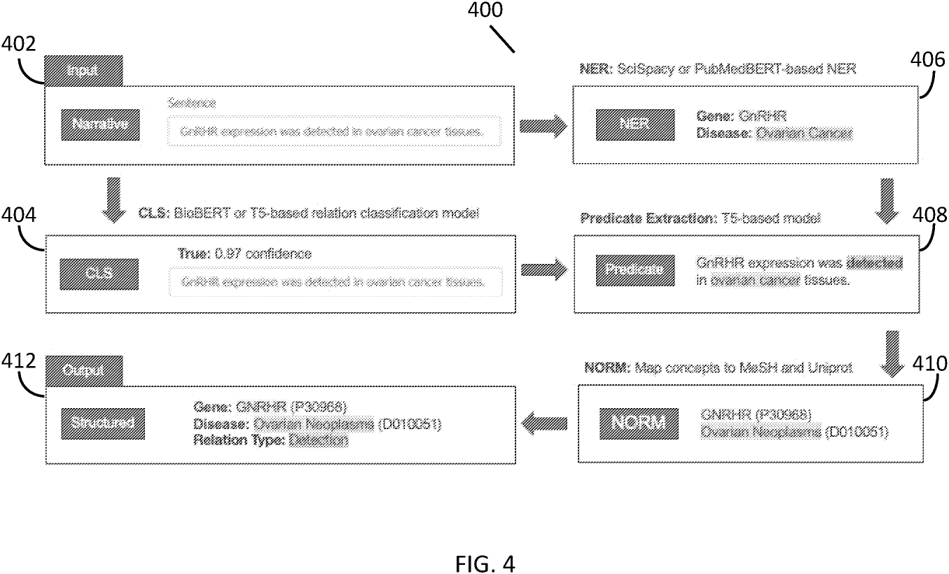

A Relation Classification component may be a component configured to identify biomedical relationships and relationship categories given text that may or may not contain biomedical named-entities. Details on binary relation classification may be found in greater detail below. In some embodiments, relation types are categorized. For a positive relationship predicted by the binary relation classifier, a multi-label or multi-class classifier with semantic role labeler may be applied to obtain relation types. Such a granularity may enable creation of triplets from biomedical texts that contain named-entities. As a non-limiting example, encoder-decoder transformers may be utilized. Encoder-decoder transformers, specifically T5, may outperform encoder-only transformers, for example, when executed on public biomedical relation extraction datasets. Methods of relation extraction described herein utilize or may be utilized in conjunction with multi-task learning to learn the shared complementary features across multiple biomedical relation extraction datasets. FIG. 4 is a diagram illustrating an example of an end-to-end triplet extraction from biomedical texts.

Accordingly, the relation classification component and/or another system component may be configured to extract triplets from text. In a preferred embodiment, a triplet extract may comprise subject, predicate, and object. In an alternate embodiment, a triplet extract may comprise subject, relation, and object. In effect, a triplet extract may implicitly contain information disclosing relationship characteristics between two entities. For example, given an input of “GnRHR expression was detected in ovarian cancer tissues,” the triplet extract may be (GnRHR, ovarian cancer, detected) based on the form (subject, object, predicate). In one embodiment, triplet extraction may include machine learning model analysis of an input, wherein the input is a sentence, and wherein the output is the subject, object, and predicate. However, in another embodiment, triplet extraction may include machine learning model analysis of an input, wherein the input includes a sentence, subject, and object, and wherein the output includes a predicate. Yet, in further embodiments, the machine learning model may be configured to analyze any suitable input and derive any suitable output. Thus, the systems and methods described herein may utilize triplet extraction comprising any combination of the previously described triplet extraction aspects.

An Entity Normalization component may be a component configured to reduce the morphological variance of extracted named-entities. Accordingly, this step may be adapted to reduce redundancy in a knowledge graph. Named-entities may be normalized, mapped, and/or grouped by unique identifiers. In various embodiments, unique identifiers are collected from publicly available knowledgebases such as Unified Medical Language System (UMLS) MetaMap and MeSH terms, and commercially available knowledgebases. However, any suitable identifiers may be utilized in entity normalization.

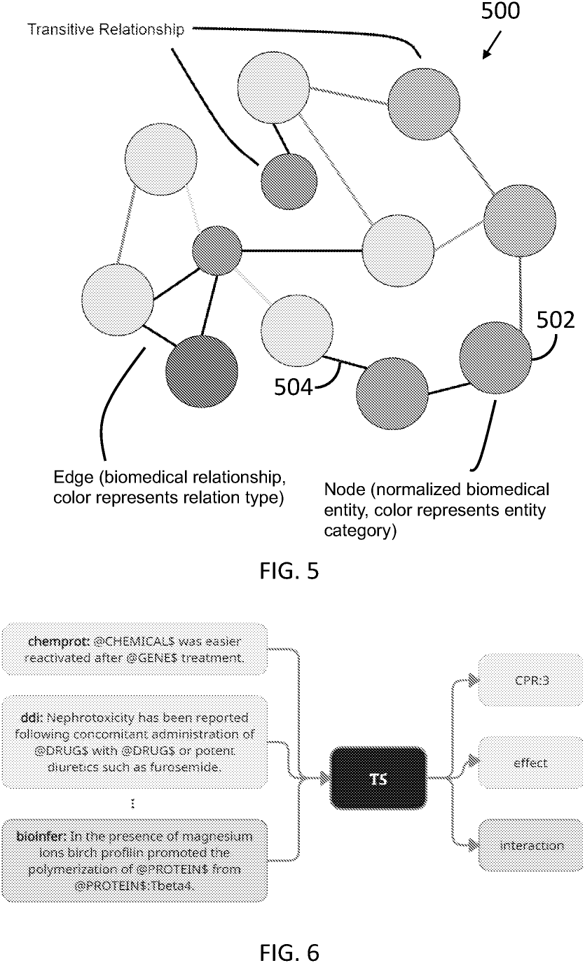

A Knowledge Graph may be configured to combine results from Named-entity recognition (NER), entity normalization, relation classification (e.g., binary, multi-class, or multi-label), and/or triplet extraction into a centralized platform. The knowledge graph may be implemented using a graph database platform. In an embodiment, the creation of the knowledge graph enables visualization and exploration of transitive relationships via a frontend web application, and to discover transitive relationships (e.g., indirect) via simple queries. In some embodiments, a knowledge discovery system (e.g., using state-of-the-art NLP models and various data sources) enables efficient recognition of hidden connections between known entities (e.g., gene-disease association). In some embodiments, relationships among nodes are estimated (e.g., via creation of knowledge graph embeddings and their use to estimate distances between nodes). FIG. 5 is a diagram illustrating a simplified example of a biomedical knowledge graph.

The biomedical relation extraction described herein may be adapted to automatically discover high-quality and semantic relations between the entities from narrative free-text. Use of encoder-decoder transformers (e.g., T5) may exhibit extensive empirical improvements over encoder-only transformers, for example, on public biomedical relation extraction datasets. Relation extraction may be configured for one or more relation extraction tasks, such as chemical-protein relation extraction, disease-protein relation extraction, drug-drug interaction, and protein-protein interaction. Yet further, multi-task fine-tuning may be utilized to evaluate and improve the correlation among major biomedical relation extraction tasks. For the purposes of this disclosure, performance may be reported using micro F-scores. In one embodiment, use of T5 and multitask learning improves the performance of the biomedical relation extraction task(s).

Unlike encoder-only transformers, which are designed to predict a single prediction for an input sequence, T5 may be configured to generate target tokens based on an encoder-decoder architecture. Therefore, T5 may perform better than in domain BERT-based models (encoder-only) such as BioBERT and PubMedBERT. Moreover, fine-tuning T5 with multi-task learning may substantially improve the RE performance compared to single task fine-tuning.

Referring to FIG. 4, triplet extraction workflow 400 may include one or more modules and/or steps. The triplet extraction workflow 400 may include an input module 402, wherein the input module 402 is configured to receive a portion of text or other suitable input, such as a sentence. In various embodiments, the input module 402 is configured to receive narrative free text as an input. However, the input module 402 may be configured to receive structured or unstructured text.

The input module 402 may be in informatic communication with a classification module 404. The classification module 404 may be configured to receive the text from the input module 402. Further, the classification module 404 may include a model (i.e., BioBERT or T5-based relation classification model) and/or other software aspect adapted to classify the text received from the input module 402. In an embodiment, such a model and/or other software aspect may be configured to evaluate the received text in terms of a confidence score. As a non-limiting example, the classification module 404 may act as a threshold evaluation, wherein, based on a confidence score, the text may or may not be determined to include the perquisite classification.

The NER module 406 may be configured to determine one or more entities based on the text received from the input module 402. In one embodiment, the NER module 406 may include a model (i.e., a SciSpacy or PubMedBERT-based NER) and/or other software aspect configured to extract one or more entities. As a non-limiting example, such a model and/or software aspect may extract a subject and object, for example, a gene (i.e., GnRHR) and a disease (i.e., Ovarian Cancer). However, the NER module 406 may be configured to extract any number and/or combination of possible entities and/or entity categories.

In an embodiment, the predicate extraction module 408 is in informatic communication with the classification module 404 and the NER module 406. However, in alternate embodiments, the workflow 400 may include one of either the classification module 404 and the NER module 406. The predicate extraction module 408 may include a model (i.e., a T5-based model) and/or other software aspect configured to extract a predicate. In such an embodiment, the predicate extraction module 408 is configured to analyze any of the input text (i.e., as received in input module 402), the classification and/or confidence score as determined in the classification module 404, and/or the entities extracted in the NER module 406.

In a further embodiment, the workflow 400 includes a normalization module 410. The normalization module 410 may receive informatic communication from the predicate extraction module 408. The normalization module 410 may include a model and/or software aspect configured to map concepts. As a non-limiting example, named-entities may be normalized, mapped, and/or grouped by unique identifiers. In such a non-limiting example, identifiers are collected from knowledgebases (i.e., UMLS, MetaMap, MeSH terms). One or more entities, for example, those previously received from the predicate extraction module 408, may be mapped to identifiers. Such a process may reduce redundancy in subsequent generation of a knowledge graph.

The workflow 400 may also include an output module 412. The output module 412 may be in informatic communication with at least the normalization module 410. In an embodiment, the output module 412 may be configured to provide a structured result, for example, a structured triplet. As a non-limiting example, the triplet may include a subject, object, and predicate, wherein the subject and/or the object are mapped to a unique identifier. The output module 412 may also structure an output, wherein said output comprises a relation type.

Referring to FIG. 5, the invention of the present disclosure may include a knowledge graph 500. In an embodiment, the knowledge graph 500 may be generated based on the NER, entity normalization, relation classification (e.g., binary, multi-class, or multi-label), and/or triplet extraction. The knowledge graph 500 may include one or more nodes 502 and one or more edges 504. A node 502 may include a normalized biomedical entity, wherein an edge 504 may represent a biomedical relationship. Accordingly, various characteristics of the nodes 502 and/or edges 504 may be modified to communicate a particular aspect to the viewer. As a non-limiting example, the color of the node 502 may be modified to indicate various entity categories. Further, as another non-limiting example, the color of the edge 504 may be modified to indicate various relation types. Yet further, the size of the node 502 may be modified to indicate characteristics of the underlying normalized biomedical entity, for example, the determined relevance, popularity, length, or other metrics. While an edge 504 may represent an adjacent relationship between two nodes 502, a transitive relationship may be formed by the indirect connection of two nodes 502. For example, as shown in FIG. 5, a first node and a second node may be in a transitive relationship if each of the first and second nodes shares an edge with a third node. The relationships among nodes described herein may be estimated (e.g., via creation of knowledge graph embeddings and their use to estimate distances between nodes). The potential modification of node 502 and edge 504 visual characteristics may be referred to herein as nodal indications and edge indications, respectively. For example, a node 502 corresponding to a first entity category may include a first nodal indication (e.g., a red color), and a node 502 corresponding to a second entity category may include a second nodal indication (e.g., a green color). In an embodiment, the size of a node 502 may refer to the frequency of the entity value associated with node 502. For example, nodal size may be a function of the frequency in which an entity value appears in a particular set of texts.

In a further embodiment, the knowledge graph 500 may be configured to receive a selection input (i.e., a mouse click from a user) corresponding to one of the plurality of nodes or the plurality of edges. Such a selection input may cause the system to generate a summary representation, for example, comprising the subject, the object, the predicate, the relation, the syntactic unit, or the original text or portions thereof, based on the selection input. Accordingly, such a summary representation may be displayed on the client device 102-106.

### Example - Encoder-Decoder Transformer Performance and Architecture

Regarding the following example, a person of ordinary skill in the art, upon reviewing the example and entirety of this disclosure, would appreciate that the techniques analyzed and compared below may be implemented in any suitable portions of the pipeline 300, workflow 400, or knowledge graph 500. Therefore, while the included example may reference particular values or instances, the techniques, methods, and components described below are not limited to their exemplary recitations.

Given an input sentence S consisting of n tokens, i.e., S= {w₁,w₂,...,wₙ} and a pair of entities (e₁,e₂) where e₁ ∈ S and e₂ ∈ S, RE models may be tasked with predicting the maximum probable label y^ from the set of labels in annotated data, y. For the purposes of this disclosure, a sentence may be referred to herein as a syntactic unit. Further, for the purposes of NER and relation extraction described herein, a syntactic unit may also refer to a paragraph, phrase, or other warranted portion of text. In yet further embodiments, the syntactic unit (and originating document thereof) may correlate to a media form other than text, for example, audio, visual, or other related media types.

### Datasets and Processing

Various benchmark datasets of RE may be analyzed between various entity types such as protein-protein, drug-drug, chemical-protein and disease-protein. Typically, a majority of relation instances are within single sentences in datasets of the aforementioned relation types. Thus, the models used herein may be configured for sentence-level relation classification. Experimental statistics of biomedical RE datasets are listed in Table 1.

TABLE 1

Statistics of the biomedical relation extraction datasets

| Dataset | Train | Dev | Test | Metrics |
|---|---|---|---|---|
| AIMed | 4938 | - | 549 | micro F1 |
| BioInfer | 8544 | - | 950 | micro F1 |
| HPRD50 | 389 | - | 44 | micro F1 |
| IEPA | 734 | - | 82 | micro F1 |
| LLL | 300 | - | 34 | micro F1 |
| DDI | 2937 | 1004 | 979 | micro F1 |
| ChemProt | 4154 | 2416 | 3458 | micro F1 |
| DrugProt | 17277 | 3765 | - | micro F1 |
| GAD | 4796 | - | 534 | micro F1 |
| EU-ADR | 318 | - | 37 | micro F1 |

Protein-protein interactions. Five benchmark datasets, namely BioInfer, AIMed, IEPA, HPRD50, and LLL, may be utilized. Such datasets may be converted to a unified format, for example, as disclosed in Pyysalo et al. (2008). Sentences that contain a pair of proteins may be selected to generate positive and negative instances. All protein-protein pairs that occur in a sentence and do not have an explicit label in aforementioned datasets may be considered as negative instances. Target named-entities may be anonymized in a sentence using a pre-defined tag, i.e., @PROTEIN$. As a non-limiting example, a sentence with two protein names is represented as “The POU domains of the @PROTEIN$ and Oct2 transcription factors mediate specific interaction with @PROTEIN$.”.

Drug-drug interactions. An existing preprocessed corpus may be utilized, such as a version of the Drug-Drug Interaction (DDI) 2013 corpus (Herrero-Zazo et al., 2013) and its corresponding train/dev/test split created by Peng et al. (2019b). Drug names may be anonymized using a tag, i.e., @DRUG$. As a non-limiting example, a sentence with a pair of drug names is represented as “Ketoconazole: @DRUG$ may inhibit both synthetic and catabolic enzymes of @DRUG$.”

Disease-protein relationships. An existing preprocessed corpus may be utilized, such as versions of the Genetic Association Database corpus (GAD) (Bravo et al., 2015) and EU-ADR datasets (van Mulligen et al., 2012). In such an embodiment, for both datasets, their corresponding train/dev/test splits created by Lee et al. (2019) may be utilized. Targeted entities may be anonymized using tags, i.e., @DISEASE$ and @GENE$. As a non-limiting example, a sentence with a pair of two entities (gene and disease in this case) is represented as “In conclusion, @GENE$ 8092C > A polymorphism may modify the associations between cumulative cigarette smoking and @DISEASE$ risk.”

Chemical-protein relationships (CPR). Datasets that contain gene-chemical relations may be utilized, e.g., ChemProt (Krallinger et al., 2017) and DrugProt (Miranda et al., 2021). For ChemProt, an existing preprocessed version and their corresponding train/dev/test split created by Peng et al. (2019b) may be used. In one embodiment, the same five classes: CPR:3, CPR:4, CPR:5, CPR:6, CPR:9 may be evaluated. For DrugProt, the standard training and development sets in the DrugProt shared task may be used and the same 13 classes: Activator, Agonist, Agonist-Inhibitor, Antagonist, Direct-Regulator, Indirect-Downregulator, Indirect-Upregulator, Inhibitor, Part-Of, Product-Of, Substrate, Substrate_Product-Of, AgonistActivator may be evaluated. In one embodiment, abstracts may be split into sentences using a Natural Language Toolkit (NLTK) and then target entities anonymized in a sentence using tags, i.e., @CHEMICAL$ and @GENE$. As a non-limiting example, a sentence with a pair of two entities (chemical and gene in this case) is represented as “During differentiation, @CHEMICAL$ promoted early expression of osteoblast transcription factors, @GENE$ and osterix.”

For the purposes of showing improved performance, in-domain BERT-based language models such as BioBERT (Lee et al., 2019) and PubMedBERT (Gu et al., 2022) may be compared with T5 (Raffel et al., 2020) and its variant SciFive (Phan et al., 2021), which is trained on biomedical texts (i.e., PubMed abstracts). For BERT-based models, a [CLS] token may be used for the classification of relations. The [CLS] representation may be fed into a SoftMax layer for a multi-way classification. For the T5-based models, the input sequence for the relation extraction task may be “Processed sentence: [s] Relation: [r]”. In one embodiment, T5 may be finetuned to generate tokens of relation types which may be the ground truth labels in training datasets.

FIG. 6 illustrates an example of multi-task fine-tuning (MTFT) utilized in RE tasks. In one embodiment, the proportional and temperature-scaled task mixing as in (Raffel et al., 2020) may be utilized for data mixture. During fine-tuning, a task-specific token (i.e., name of the dataset) may be prepended to the input sequence. For the purposes of this disclosure, multi-task fine-tuning may include any multi-task learning aspects known to those of ordinary skill in the art. Accordingly, such a multi-task learning model may receive standard machine learning model input, wherein the learned features may be utilized to yield multiple output values, wherein said multiple output values may link to each of the anticipated tasks. Therefore, multi-task learning models and associated MTFT may be beneficial in scenarios where solving a first task conveys information that may be valuable in resolving a second task related to the first task.

In various embodiments, the following may be utilized: the BioBERT (v1.1base-PubMed), PubMedBERT, T5-base, and SciFive (SciFive-base-Pubmed). Models may be trained with a batch size of 16 and maximum sequence length of 300 tokens for 10 epochs using single GPU (16 GB VRAM) on a platform such as Amazon SageMaker. An optimization algorithm such as an Adam optimizer with a learning rate of 1e-5 may be used.

Table 2 show the results of T5-based models compared to the in-domain and SOTA BERT-based models (pretrained on biomedical text) on ten benchmarking biomedical RE datasets, listed in Table 1. The micro F1 scores obtained by T5 and its variant SciFive (pretrained on PubMed abstracts) may be compared to those of the BioBERT and PubMedBERT. On average (micro), T5 which, in such an instance, may only be pretrained on the general domain corpus, obtained a higher F1 score than BioBERT and PubMedBERT. Further, in such an example, T5 achieved the highest F1 scores on 5 out of 10 biomedical RE datasets. In various embodiments, models using biomedical text in pre-training generally perform better than models pre-trained on general domain corpus.

TABLE 2

Biomedical relation extraction test results

| Relation | Datasets | BioBERT | PubMedBERT | T5 | T5-SciFive T5-MTFT | |
|---|---|---|---|---|---|---|
| | AIMed | 92.36 | 93.31 | 94.35 | 94.17 | 93.62 |
| | BioInfer | 95.97 | 94.59 | 95.36 | 95.89 | 95.16 |
| Protein-protein | HPRD50 | 85.45 | 90.56 | 84.09 | 90.90 | 95.95 |
| | IEPA | 86.58 | 86.46 | 87.80 | 87.80 | 90.24 |
| | LLL | 88.24 | 100.0 | 97.05 | 94.11 | 97.05 |
| Drug-drug | DDI | 89.67 | 90.69 | 91.01 | 90.60 | 91.83 |
| Chemical-protein | ChemProt | 90.11 | 91.64 | 90.45 | 92.39 | 96.56 |
| | DrugProt | 88.69 | 89.40 | 88.71 | 89.56 | 89.37 |
| Disease-protein | GAD | 79.91 | 80.87 | 81.46 | 81.27 | 80.71 |
| | EU-ADR | 57.42 | 64.63 | 78.38 | 75.67 | 83.78 |
| Average score | | 85.44 | 88.22 | 89.47 | 89.23 | 91.42 |

MTFT may also be utilized to impact benchmark biomedical RE tasks, i.e., drug-drug interaction, protein-protein interaction, chemical-protein relation extraction, and disease-protein relation extraction. On average, performance improves when using MTFT (as shown in Table 2, an improvement of 1.95 F-score over the best single performing model). For instance, on the ChemProt dataset, T5-MTFT was able to achieve significant performance improvement of 6.11 and 6.45 F-score points over T5 and BioBERT respectively. While overall results indicate that MTFT provides improved RE performance on the four biomedical RE tasks (i.e., tasks with clear knowledge transfer), a slight drop may be observed in the performance on some datasets such as AIMed, BioInfer, and GAD. In MTFT, in addition to the sample size of each task, the difficulty of the task/dataset may have an impact on the overall performance (for example, the model may underfit or overfit datasets and then evaluate on the test set for each dataset).

Error Analysis. A manual analysis of the test sets was performed, wherein the best performing model predicted an incorrect label.

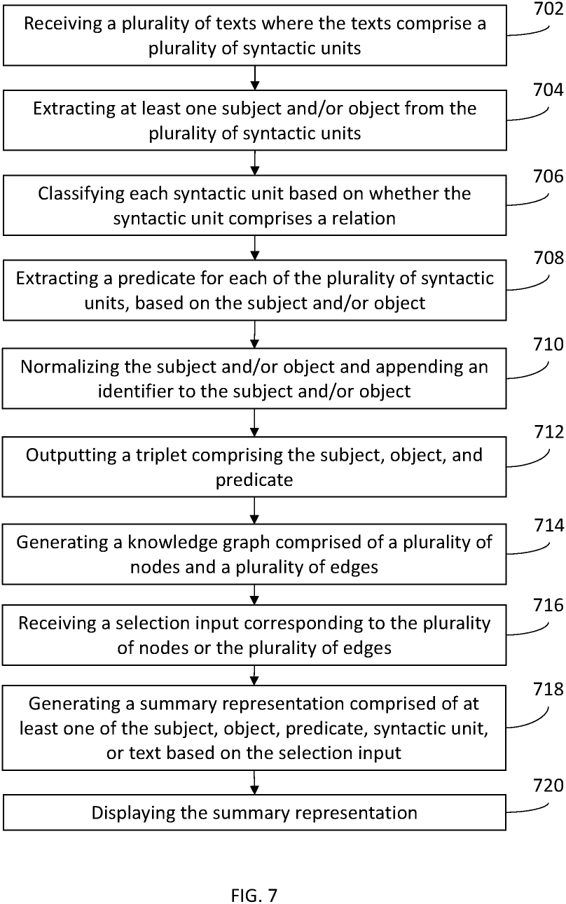

FIG. 7 illustrates an embodiment of a triplet and graphical visualization generation workflow 700. At 702, a plurality of texts may be received wherein the plurality of texts comprise a plurality of syntactic units. For example, the plurality of texts may refer to a collection of medical journal abstracts or other documents. In such an example, the syntactic units may refer to syntactic components of the text, such as sentences. Accordingly, in one embodiment, the workflow 700, and triplet extraction generally, may be an iterative process configured to analyze each sentence of a body of text. At 704, at least one subject and/or at least one object may be extracted from each of the syntactic units. In such an embodiment, at 704, a NER model may be configured to extract any number of subjects and/or any number of objects. Such a model may be configured to determine the entity type for the extracted subjects and objects. As a non-limiting example, the NER model may extract one subject and one object from a particular syntactic unit, wherein the NER model determines that the subject has a “gene” entity type and the object has a “disease” entity type. Accordingly, for the purposes of the workflow 700, the subject, the object, the corresponding entity types (i.e., gene and disease), and entity values (i.e., GnRHR and Ovarian Cancer) may be communicated throughout the workflow 700. In an embodiment, at 704, a NER model may be configured to return an error, populate an empty entity value/type, or cease the workflow if one or more conditions are met. For example, such conditions may include instances when a subject or an object is not determined. Alternatively, such conditions may include instances when a particular number of subjects and/or objects are determined (i.e., two subjects and three objects). However, in various embodiments, the NER model may be configured to output any determined results to the predicate extraction model, regardless of the NER results characteristics.

In an embodiment, at 706, a classification model may evaluate each syntactic unit to determine whether the syntactic unit comprises a relation. The classification model may determine whether a positive and/or negative relationship exists in a particular syntactic unit. For example, the classification model may be a binary relation classifier, a multi-label or multi-class classifier with a semantic role labeler applied to obtain relation types. In a further embodiment, the classification model may be configured such that the classification model returns a positive status as a function of a predetermined threshold confidence score.

In an embodiment, at 708, a predicate extraction model may receive input from both the classification model and the NER model. In an embodiment, the predicate extraction model may be configured to extract a predicate from a particular syntactic unit based on the subject, the object, and/or the content of the syntactic unit. If the predicate cannot be determined and/or if the predicate extraction model has received an incomplete or noncompliant classification, subject, object, and/or syntactic unit, the predicate extraction model may return an error, populate an empty predicate value/type, or cease the workflow.

In a further embodiment, at 710, the subject and/or object of a particular syntactic unit may be normalized, at least in part, by appending the subject and object with unique identifiers. For example, each subject and/or object may be of a particular entity type/category and entity value. Accordingly, each entity value may be associated with a corresponding identifier. Further, normalization may include normalizing the “content” of the subject and/or object. For example, as shown in FIG. 4, the normalization module 410 may be configured to convert entity values (i.e., GnRHR and Ovarian Cancer) to normalized entity values (i.e., GNHRH and Ovarian Neoplasms). Therefore, colloquialisms, spelling variations, and other variants may be normalized and redundancy may be reduced.

In an embodiment, at 712, a triplet may be outputted comprising the subject and corresponding entity type (i.e., Gene) and value (i.e., GNRHR), the object and corresponding entity type (i.e., Disease) and value (i.e., Ovarian Neoplasms), and the predicate and corresponding predicate type (i.e., Relation Type) and value (i.e., Detection) determined and normalized in steps 702-710.

In yet a further embodiment, at 714, a knowledge graph may be generated comprising a plurality of nodes and a plurality of edges.

As depicted in 716, a selection input may be received, wherein the selection input corresponds to a user’s selection of a particular node or edge. At 718, a summary representation may be generated comprising any one of the subject, object, predicate, syntactic unit, original text or a section thereof, and/or any combination or number of pre-normalized or normalized information determined or received in steps 702-714. In a further embodiment, the summary representation may be presented to a user, for example, on a client device.

In an aspect of this disclosure, a computer-implemented method comprises the steps of receiving a plurality of texts, each of the plurality of texts comprising a plurality of syntactic units; extracting, via a NER model, for each of the plurality of syntactic units, at least one subject and/or at least one object; and classifying, via a classification model, for each of the plurality of syntactic units, whether each of the plurality of syntactic units comprises a relation. The method may further comprise the steps of extracting, via a predicate extraction model, for each of the plurality of syntactic units, based on the at least one subject, the at least one object, and the corresponding syntactic unit, a predicate; normalizing, for each of the plurality of syntactic units, the at least one subject and the at least one object, and appending a subject identifier and an object identifier to the at least one subject and the at least one object, respectively; and outputting, for each of the plurality of syntactic units, a triplet comprising the at least one subject, the at least one object, and the predicate.

In a further embodiment, the method may comprise the step of generating, based on the triplets derived from the plurality of texts, a knowledge graph comprising a plurality of nodes and a plurality of edges, wherein each of the plurality of nodes corresponds to at least one subject or at least one object of the triplets derived from the plurality of texts, wherein each of the plurality of edges corresponds to a relationship type, and wherein the relationship type is based on at least the predicate of each triplet derived from the plurality of texts.

In an embodiment, each of the plurality of nodes are configurable in a plurality of nodal indications, wherein each of the plurality of nodal indications correspond to one of a plurality of entity categories. Further, each of the plurality of edges may be configurable in a plurality of edge indications, wherein each of the edge indications may correspond to one of a plurality of relationship categories. In an embodiment, the method comprises the steps of receiving, via a client device, a selection input corresponding to one of the plurality of nodes or the plurality of edges; generating a summary representation comprising at least one of the subject, the object, the predicate, the syntactic unit, or the text, based on the selection input; and displaying, via the client device, the summary representation.

In an aspect of the invention of the present disclosure, the predicate extraction model is an encoder-decoder model. Further, the encoder-decoder model may be tuned via a multi-task fine-tuning. In one embodiment, the encoder-decoder model is trained on a plurality of biomedical texts. In various embodiments, the classification model is an encoder-only model or an encoder-decoder model.

The invention of the present disclosure may be a system comprising a server comprising at least one server processor, at least one server database, at least one server memory comprising computer-executable server instructions which, when executed by the at least one server processor, cause the server to receive a plurality of texts, each of the plurality of texts comprising a plurality of syntactic units; and extract, via a NER model, for each of the plurality of syntactic units, at least one subject and/or at least one object. In a further embodiment, the computer-executable server instructions which, when executed by the at least one server processor, cause the server to classify, via a classification model, for each of the plurality of syntactic units, whether each of the plurality of syntactic units comprises a relation; extract, via a predicate extraction model, for each of the plurality of syntactic units, based on the at least one subject, the at least one object, and the corresponding syntactic unit, a predicate; normalize, for each of the plurality of syntactic units, the at least one subject and the at least one object, and append a subject identifier and an object identifier to the at least one subject and the at least one object, respectively; and output, for each of the plurality of syntactic units, a triplet comprising the at least one subject, the at least one object, and the predicate.

The computer-executable server instructions which, when executed by the at least one server processor, may further cause the server to generate, based on the triplets derived from the plurality of texts, a knowledge graph comprising a plurality of nodes and a plurality of edges, wherein each of the plurality of nodes corresponds to at least one subject or at least one object of each of the triplets derived from the plurality of texts, wherein each of the plurality of edges corresponds to a relationship type, and wherein the relationship type is based on at least the predicate of each of the triplets derived from the plurality of texts. In an embodiment, each of the plurality of nodes are configurable in a plurality of nodal indications, wherein each of the plurality of nodal indications correspond to one of a plurality of entity categories. In a further embodiment, each of the plurality of edges are configurable in a plurality of edge indications, wherein each of the edge indications correspond to one of a plurality of relationship categories.

The system may further comprise a client device in bidirectional communication with the server, the client device comprising at least one device processor, at least one display, at least one device memory comprising computer-executable device instructions which, when executed by the at least one device processor, may cause the client device to receive, via the client device, a selection input corresponding to one of the plurality of nodes or the plurality of edges; and display, via the client device, a summary representation. The computer-executable server instructions which, when executed by the at least one server processor, may further cause the server to generate the summary representation comprising at least one of the subject, the object, the predicate, the syntactic unit, or the text, based on the selection input.

In an embodiment, the predicate extraction model is an encoder-decoder model. The encoder-decoder model may be tuned via multi-task fine-tuning. Further, the encoder-decoder model may be trained on a plurality of biomedical texts. In various embodiments, the classification model is an encoder-only model or an encoder-decoder model.

The invention of the present disclosure may be a non-transitory computer readable medium having instructions stored thereon that, when executed by a processing device, cause the processing device to carry out an operation of triplet extraction and visualization generation between a server and a client device, the operation comprising receiving a plurality of texts, each of the plurality of texts comprising a plurality of syntactic units; and extracting, via a NER model, for each of the plurality of syntactic units, at least one subject and/or at least one object. In an embodiment, the operation further comprises classifying, via a classification model, for each of the plurality of syntactic units, whether each of the plurality of syntactic units comprises a relation; extracting, via a predicate extraction model, for each of the plurality of syntactic units, based on the at least one subject, the at least one object, and the corresponding syntactic unit, a predicate; and normalizing, for each of the plurality of syntactic units, the at least one subject and the at least one object, and appending a subject identifier and an object identifier to the at least one subject and the at least one object, respectively. In yet a further embodiment, the operation comprises outputting, for each of the plurality of syntactic units, a triplet comprising the at least one subject, the at least one object, and the predicate.

In a further embodiment, the operation comprises generating, based on the triplets derived from the plurality of texts, a knowledge graph comprising a plurality of nodes and a plurality of edges, wherein each of the plurality of nodes corresponds to at least one subject or at least one object of each of the triplets derived from the plurality of texts, wherein each of the plurality of edges corresponds to a relationship type, and wherein the relationship type is based on at least the predicate of each of the triplets derived from the plurality of texts. In an embodiment, each of the plurality of nodes are configurable in a plurality of nodal indications, wherein each of the plurality of nodal indications correspond to one of a plurality of entity categories. In another embodiment, each of the plurality of edges are configurable in a plurality of edge indications, wherein each of the edge indications correspond to one of a plurality of relationship categories.

In yet a further embodiment, the operation further comprises receiving, via the client device, a selection input corresponding to one of the plurality of nodes or the plurality of edges; generating a summary representation comprising at least one of the subject, the object, the predicate, the syntactic unit, or the text, based on the selection input; and displaying, via the client device, the summary representation. In an embodiment, the predicate extraction model is an encoder-decoder model. In a further embodiment, the encoder-decoder model is tuned via a multi-task fine-tuning. The encoder-decoder model may be trained on a plurality of biomedical texts. In various embodiments, the classification model is an encoder-only model or an encoder-decoder model.

Various elements, which are described herein in the context of one or more embodiments, may be provided separately or in any suitable subcombination. Further, the processes described herein are not limited to the specific embodiments described. For example, the processes described herein are not limited to the specific processing order described herein and, rather, process blocks may be re-ordered, combined, removed, or performed in parallel or in serial, as necessary, to achieve the results set forth herein.

It will be further understood that various changes in the details, materials, and arrangements of the parts that have been described and illustrated herein may be made by those skilled in the art without departing from the scope of the following claims.

All references, patents and patent applications and publications that are cited or referred to in this application are incorporated in their entirety herein by reference. Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

Finally, other implementations of the disclosure will be apparent to those skilled in the art from consideration of the specification and practice of the disclosure disclosed herein. It is intended that the specification and examples be considered as exemplary only, with a true scope and spirit of the disclosure being indicated by the following claims.

What is claimed is:

1. A computer-implemented method, comprising the steps of:
receiving a plurality of texts, each of the plurality of texts comprising a plurality of syntactic units;
extracting, via a Named-entity recognition (NER) model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate;
normalizing, for each of the plurality of syntactic units, the at least one subject and the at least one object, and appending a subject identifier and an object identifier to the at least one subject and the at least one object, respectively; and
outputting, for each of the plurality of syntactic units, a triplet comprising the at least one subject, the at least one object, and the predicate.

2. The computer-implemented method of claim 1, further comprising the step of:
generating, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

3. The computer-implemented method of claim 2, wherein each of the plurality of nodes are configurable in a plurality of nodal indications, wherein each of the plurality of nodal indications correspond to one of a plurality of entity categories.

4. The computer-implemented method of claim 2, wherein each of the plurality of edges are configurable in a plurality of edge indications, wherein each of the edge indications correspond to one of a plurality of relationship categories.

5. The computer-implemented method of claim 2, further comprising the steps of:
receiving, via a client device, a selection input corresponding to one of the plurality of nodes or the plurality of edges;
generating a summary representation comprising at least one of the subject, the object, the predicate, the syntactic unit, or the text, based on the selection input; and
displaying, via the client device, the summary representation.

6. The computer-implemented method of claim 1, wherein the predicate extraction model is an encoder-decoder model.

7. The computer-implemented method of claim 6, wherein the encoder-decoder model is tuned via a multi-task fine-tuning.

8. The computer-implemented method of claim 6, wherein the encoder-decoder model is trained on a plurality of biomedical texts.

9. The computer-implemented method of claim 1, wherein the classification model is an encoder-only model.

10. The computer-implemented method of claim 1, wherein the classification model is an encoder-decoder model.

11. A system, comprising:
a server comprising at least one server processor, at least one server database, at least one server memory comprising computer-executable server instructions which, when executed by the at least one server processor, cause the server to:
receive a plurality of texts, each of the plurality of texts comprising a plurality of syntactic units;
extract, via a NER model, for each of the plurality of syntactic units, at least one subject and/or at least one object;
classify, via a classification model, for each of the plurality of syntactic units, whether each of the plurality of syntactic units comprises a relation;
extract, via a predicate extraction model, for each of the plurality of syntactic units, based on the at least one subject, the at least one object, and the corresponding syntactic unit, a predicate;
normalize, for each of the plurality of syntactic units, the at least one subject and the at least one object, and appending a subject identifier and an object identifier to the at least one subject and the at least one object, respectively;
output, for each of the plurality of syntactic units, a triplet comprising the at least one subject, the at least one object, and the predicate.

12. The system of claim 11, the computer-executable server instructions which, when executed by the at least one server processor, further cause the server to:
generate, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

13. The system of claim 12, wherein each of the plurality of nodes are configurable in a plurality of nodal indications, wherein each of the plurality of nodal indications correspond to one of a plurality of entity categories.

14. The system of claim 12, wherein each of the plurality of edges are configurable in a plurality of edge indications, wherein each of the edge indications correspond to one of a plurality of relationship categories.

15. The system of claim 12, further comprising:
a client device in bidirectional communication with the server, the client device comprising at least one device processor, at least one display, at least one device memory comprising computer-executable device instructions which, when executed by the at least one device processor, cause the client device to:
receive, via the client device, a selection input corresponding to one of the plurality of nodes or the plurality of edges; and
display, via the client device, a summary representation, and
the computer-executable server instructions which, when executed by the at least one server processor, further cause the server to:
generate the summary representation comprising at least one of the subject, the object, the predicate, the syntactic unit, or the text, based on the selection input.

16. The system of claim 11, wherein the predicate extraction model is an encoder-decoder model.

17. The system of claim 16, wherein the encoder-decoder model is tuned via multi-task fine-tuning.

18. The system of claim 16, wherein the encoder-decoder model is trained on a plurality of biomedical texts.

19. The system of claim 11, wherein the classification model is an encoder-only model.

20. The system of claim 11, wherein the classification model is an encoder-decoder model.

21. A non-transitory computer readable medium having instructions stored thereon that, when executed by a processing device, cause the processing device to carry out an operation of triplet extraction and visualization generation between a server and a client device, the operation comprising:
receiving a plurality of texts, each of the plurality of texts comprising a plurality of syntactic units;
extracting, via a NER model, for each of the plurality of syntactic units, at least one subject and/or at least one object;
classifying, via a classification model, for each of the plurality of syntactic units, whether each of the plurality of syntactic units comprises a relation;
extracting, via a predicate extraction model, for each of the plurality of syntactic units, based on the at least one subject, the at least one object, and the corresponding syntactic unit, a predicate;
normalizing, for each of the plurality of syntactic units, the at least one subject and the at least one object, and appending a subject identifier and an object identifier to the at least one subject and the at least one object, respectively; and
outputting, for each of the plurality of syntactic units, a triplet comprising the at least one subject, the at least one object, and the predicate.

22. The non-transitory computer readable medium of claim 21, the operation further comprising:
generating, based on the triplets derived from the plurality of texts, a knowledge graph comprising a plurality of nodes and a plurality of edges,
wherein each of the plurality of nodes corresponds to at least one subject or at least one object of each of the triplets derived from the plurality of texts,
wherein each of the plurality of edges corresponds to a relationship type, and
wherein the relationship type is based on at least the predicate of each of the triplets derived from the plurality of texts.

23. The non-transitory computer readable medium of claim 22, wherein each of the plurality of nodes are configurable in a plurality of nodal indications, wherein each of the plurality of nodal indications correspond to one of a plurality of entity categories.

24. The non-transitory computer readable medium of claim 22, wherein each of the plurality of edges are configurable in a plurality of edge indications, wherein each of the edge indications correspond to one of a plurality of relationship categories.

25. The non-transitory computer readable medium of claim 22, the operation further comprising:
receiving, via the client device, a selection input corresponding to one of the plurality of nodes or the plurality of edges;
generating a summary representation comprising at least one of the subject, the object, the predicate, the syntactic unit, or the text, based on the selection input; and
displaying, via the client device, the summary representation.

26. The non-transitory computer readable medium of claim 21, wherein the predicate extraction model is an encoder-decoder model.

27. The non-transitory computer readable medium of claim 26, wherein the encoder-decoder model is tuned via a multi-task fine-tuning.

28. The non-transitory computer readable medium of claim 26, wherein the encoder-decoder model is trained on a plurality of biomedical texts.

29. The non-transitory computer readable medium of claim 21, wherein the classification model is an encoder-only model.

30. The non-transitory computer readable medium of claim 21, wherein the classification model is an encoder-decoder model.

31. A non-transitory computer readable medium having instructions stored thereon that, when executed by a processing device, cause the processing device to carry out an operation of triplet extraction and visualization generation between a server and a client device, the operation comprising:
receiving a plurality of texts, each of the plurality of texts comprising a plurality of syntactic units;
extracting, for each of the plurality of syntactic units, at least one subject and/or at least one object;
classifying, for each of the plurality of syntactic units, whether each of the plurality of syntactic units comprises a relation;
extracting, for each of the plurality of syntactic units, a predicate; and
outputting, for each of the plurality of syntactic units, a triplet comprising the at least one subject, the at least one object, and the predicate.
